(19) United States
(12) Patent Application Publication
(10) Pub. No.: US 2014/0330223 A1
(43) Pub. Date: Nov. 6, 2014

(54) TRANSDERMAL THERAPEUTIC SYSTEM FOR ADMINISTERING FENTANYL OR AN ANALOGUE THEREOF

(71) Applicant: Acino AG, Miesbach (DE)

(72) Inventors: Björn Schurad, Munchen (DE); Sonja Rak, Unterhaching (DE)

(21) Appl. No.: 14/359,676

(22) PCT Filed: Nov. 22, 2012
(86) PCT No.: PCT/EP2012/073314

§ 371 Date: May 21, 2014

(30) Foreign Application Priority Data
Nov. 30, 2011 (EP) 11191254.9

(51) Int. Cl. A61K 9/70; B32B 37/26; B32B 38/00; A61K 31/4468

(52) U.S. Cl. CPC A61K 9/7053; A61K 31/4468; B32B 37/26; B32B 38/0004; B32B 2037/268; B32B 2556/00; B32B 2323/00
USPC 604/290; 604/307; 156/250

(57) ABSTRACT

According to the invention a transdermal therapeutic system for administering fentanyl or an analogue thereof through the skin is provided that has a pressure-sensitive adhesive matrix layer containing a mixture of two polyisobutylenes with specific storage moduli.

Object of the present application is a system for the transdermal administration of fentanyl or an analogue thereof for therapeutic purposes. The transdermal therapeutic system (TTS, active ingredient patch) is characterized by an excellent combination of the properties relevant for such a system, in particular sufficient adhesive capacity and cutaneous tolerance over a long period of time, so that a wearing time of the system of at least 3 days is ensured, the absence of cold flow, low active ingredient load, and outstanding active ingredient release.

Fentanyl and its analogues, in particular alfentanil, carfentanil, lofentanil, remifentanil, sufentanil, trefentanil, and related compounds are potent synthetic opiates. Fentanyl and its analogues are highly efficacious and are rapidly metabolized. A problem with these compounds is their relatively narrow therapeutic index. When the threshold values are exceeded undesired side effects occur, in particular impairment of respiration what can—unless suitable countermeasures are taken—cause death. The active ingredients are relatively expensive and there is a high risk of abuse. That's why fentanyl patches on the one hand have to ensure a very precisely controlled release of the active ingredient and on the other hand the product should be designed such that the active ingredient cannot be removed easily out of it for purposes of abuse. Generally, the patches are intended to be used for at least three days and during this period of time must sufficiently adhere to the skin.

Typically, an active ingredient patch is a small adherent bandage containing the active ingredient to be delivered. These bandages can have various forms and sizes. The simplest type is an adhesive monolith comprising an active ingredient stock (reservoir) on a carrier. Typically, the reservoir is formed of the active ingredient in a pharmaceutically acceptable pressure-sensitive adhesive. However, it can also be formed of a non (or poor) adhering material the skin-contacting surface of which is provided with a thin layer of a suitable adhesive.

More complex patches are multiple laminates or patches having an active ingredient stock (which can optionally be present solved in a liquid), wherein a membrane controlling the release of the active ingredient can be arranged between the reservoir and the skin-contacting adhesive. This membrane is for the control and optionally reduction of the effects of variations of the skin permeability by lowering the rate of delivery in vitro and in vivo of the active ingredient from the patch.

A transdermal patch can contain the active ingredient either completely dissolved in the stock or the stock can contain an excess of undissolved active ingredient (depot patch). However, the presence of undissolved active ingredient or other constituents in a patch can cause stability and other problems in storage as well as in use. Also a difficulty is that it has to be ensured that active ingredient dissolves sufficiently fast enough from the solid depot to replace the delivered active ingredient. In the state of the art, active ingredient patches the reservoir of which has solid active ingredient particles are often considered to be detrimental.

Various transdermal patches for the administration of fentanyl are known from the state of the art. WO 02/074286 describes a transdermal patch having a reservoir containing fentanyl wherein the reservoir has a polymeric composition, preferably polyacrylate, in a uniform phase state which is free of undissolved active ingredient. Here, a supersaturation should explicitly be avoided.

There are many experiments to prepare fentanyl patches also on the basis of a matrix layer of polyisobutylene. First, such experiments are already described in the basic patent regarding fentanyl patches U.S. Pat. No. 4,588,580. This printed matter discloses a transdermal therapeutic system with a polyisobutylene matrix and mineral oil which contains a 2% load of fentanyl as undissolved solid. However, in practice the system has disadvantages and in the period following the development departed from polyisobutylene matrices and if any polyisobutylene matrices were used the attempt was made to completely solve the active ingredient in the polyisobutylene matrix.

A transdermal therapeutic system with a polyisobutylene matrix is described in Roy et al., Journal of Pharmaceutical Sciences, Vol. 85, No. 5, May 1996, pp 491 to 495. It is shown that with concentrations of fentanyl in the polyisobutylene matrix of more than 4% active ingredient is precipitating and this Roy et al. obviously considered negative. For patches with a low active ingredient loading of fentanyl Roy et al. suggest a polysilicone patch and not polyisobutylene patches.

Correspondingly, in US 2007/0009588 and US 2006/0013865 polyisobutylene matrices are suggested in case of which care has to be taken that the active ingredient is present completely dissolved in the polyisobutylene matrix. Occurring of crystals in the matrix is considered to be negative. The polyisobutylene matrices contain polyisobutylenes of different molecular weights and a mineral oil that preferably is a liquid paraffin or a styrene-isoprene-styrene block-polymer.

DE 198 37 902 discloses transdermal therapeutic systems on the basis of polyisobutylene particularly suitable for administering clonidine, however, among the active ingredients mentioned there fentanyl is also found. Examples of fentanyl patches are not found in this printed matter, an indication that in the patches disclosed there the active ingredient should be present as a solid is also not found in this printed matter. The printed matter does not contain in vivo studies on the release of the active ingredient from the patches. The polyisobutylene layer of these patches contains at least 5% by weight of a filler.

In WO 2009/130039 transdermal therapeutic systems for administering fentanyl or an analogue thereof are described on which on a back layer a polyisobutylene layer is applied that contains the active ingredient and has a content of gel-forming agent of at most 4% by weight. With these patches a longer and more uniform release is to be achieved than with the known fentanyl patches.

EP 0 272 987 also discloses a patch for administering active ingredients wherein a specifically designed support layer is considered to be relevant. In one example of the printed matter a fentanyl patch is disclosed that contains two active ingredient layers wherein the one active ingredient layer represents a polyisobutylene layer and the other a polydimethyl siloxane layer. The printed matter discloses that the active ingredient flow from the polyisobutylene layer is substantially lower than from the polydimethyl siloxane layer.

WO 2011/029948 discloses a transdermal therapeutic system for administering fentanyl or an analogue of the fentanyl through the skin in which the matrix layer consists of two layers each containing a polyisobutylene of another composition. The constitution of the patches is relatively complex due to the multilayer coating.

US 2011/0020426 discloses a method for the preparation of an adhesive composition from a polyisobutylene of high molecular weight and a polyisobutylene of a lower molecular weight. Said adhesive composition is suitable for patches for administering active ingredient. The active ingredients are not particularly limited and fentanyl is disclosed as one active ingredient from a long list of active ingredients. None of the examples relates to a fentanyl patch.

EP 1 625 845 discloses a transdermal therapeutic system for administering fentanyl from an adhesive layer of two types of polyisobutylene of different molecular weights. The adhesive layer further contains a tackifier and an organic liquid that can be polybutene. As in US 2007/0009588 and US 2006/0013865 EP 1 625 845 considers as substantial that the fentanyl is completely dissolved in the adhesive layer. Accordingly, the tackifier and the organic liquid are selected to ensure a complete dissolution of the fentanyl in the adhesive layer. Preferably, the organic liquid is a mixture of a fatty acid alkyl ester and a branched long-chain alcohol, since in this mixture the solubility of the fentanyl is the best.

It is a very demanding problem to provide a transdermal therapeutic system for the administration of fentanyl or an analogue thereof that combines all properties relevant in practice in an advantageous manner. Such a transdermal therapeutic system not only has to ensure a sufficient active ingredient release over the entire duration of use, but additionally also a sufficient adhesive capacity and cutaneous tolerance in order that the usual duration of use for fentanyl and analogues thereof of typically three days is ensured. Furthermore, no appreciable cold flow must occur in the transdermal therapeutic system. In case of such a cold flow the transdermal therapeutic system virtually becomes useless during storage. Finally, the patch should be easy and cost-effective to prepare, make do with an active ingredient load which is as little as possible and have a content of residual active ingredients after use which is as low as possible.

The polyisobutylene-based patches suggested in the prior art meet parts of the forgoing desired properties. For example, the patch having a matrix layer of two polyisobutylenes of different molecular weights described in EP 1 625 845 exhibits a satisfactory release of the active ingredient, however, the adhesive performance of the patches is not satisfactory. Obviously, with an insufficient adhesive performance of the patch the use over a longer period of time, e.g. three days or more, is not reliably possible. Also, the amount of active ingredient that is introduced into the patch is too small for a multi-day patch for the treatment of severe pain. In WO 2011/029948 there was found a way to improve the adhesive properties of the polyisobutylene-based fentanyl patches and at the same time to achieve an outstanding active ingredient release. Into the patches an amount of active ingredient can be introduced that is also sufficient to treat severe pain over several days. However, the patches disclosed there tend to show cold flow and the constitution of the patches with two stacked pressure-sensitive adhesive layers of different polyisobutylenes is relatively complex what makes the preparation of the patch expensive and costly.

Moreover, the known patches show a significant content of residual active ingredients after use. According to H. G. Kress et al., European Journal of Pharmaceutics and Biopharmaceutics, 75(2010), 225-231 the measured content of residual fentanyl in commercially available Matrifen® and Durogesic DTrans® Patches on average is 82.3% or 52.3%, respectively, although their release is bioequivalent. Accordingly, the total content in Durogesic DTrans® is significantly higher before use. However, a residual content of active ingredient after use in the range of from 50-80% not only from economic aspects is disadvantageous, but in particular represents a significant risk of abuse.

Thus, there is the further need for a patch based on polyisobutylenes for administering fentanyl or an analogue thereof that has the aforementioned advantageous combination of properties, that is, not only provides a superior release of the active ingredient, but also very good adhesive properties and superior wearing comfort (each for three days or more), while showing no cold flow during storage and being easily and inexpensively to be prepared with low active ingredient input and a low content of residual active ingredient after use.

Now, the inventors of the present application surprisingly have found that the temperature dependency of the viscoelastic behavior of the polyisobutylenes of the matrix layer of a transdermal therapeutic system is of decisive importance for the relevant properties of the transdermal therapeutic system, in particular for the combination of properties a) good adhesive property, b) reduction of cold flow, c) high active ingredient release over several days, and d) low content of residual active ingredient after use. It was found that transdermal therapeutic systems in which the matrix layer is constituted of polyisobutylenes the viscoelastic properties of which exhibit a certain temperature dependency have the combination of the aforementioned superior properties, in particular when the matrix layer also contains a permeation enhancer and a tackifier. It has surprisingly be found that in particular the temperature dependency of the storage modulus of the polyisobutylenes present in the matrix layer plays an important role to prevent cold flow, at the same provide a high adhesive capacity with very good cutaneous tolerance over a period of at least three days, and still ensure a sufficient release of the active ingredient and low content of residual active ingredient. A complex multi-layer constitution as it is demanded for example in WO 2011/029948 is not required in the transdermal therapeutic systems according to the invention.

Thus, the present application relates to a transdermal therapeutic system for administering an active ingredient through the skin comprising:

a) a back layer,

b) a pressure-sensitive adhesive matrix layer containing the active ingredient; and

c) a stripping layer (release liner);

wherein the active ingredient is fentanyl or an analogue of the fentanyl selected from alfentanil, lorfentanil, lofentanil, remifentanil, and trefentanil or a salt of one of these active ingredients and

wherein the matrix layer as the pressure-sensitive adhesive polymer contains a mixture of a polyisobutylene A and a polyisobutylene B,

wherein the polyisobutylene A has a storage modulus G′ the value of which is substantially constant in the temperature range of 10° C. to 40° C. and

wherein the polyisobutylene B has a storage modulus G′ the value of which continuously decreases with increasing temperature in the temperature range of 10° C. to 40° C.,

wherein the storage modulus G′ is measured in the linear viscoelastic range at a frequency of 10 rad/sec using a rheometer with parallel plate geometry and parallel plates; and

wherein the pressure-sensitive adhesive matrix layer contains undissolved active ingredient in the form of active ingredient particles.

The transdermal therapeutic system according to the invention as the active ingredient contains fentanyl or an analogue of the fentanyl selected from alfentanil, carfentanil, lofentanil, remifentanil, and trefentanil or a salt of one of these active ingredients. Most preferably, the active ingredient is fentanyl. In the following, the invention is explained substantially by way of the active ingredient fentanyl. However, the embodiments correspondingly apply also to the given analogues of the fentanyl.

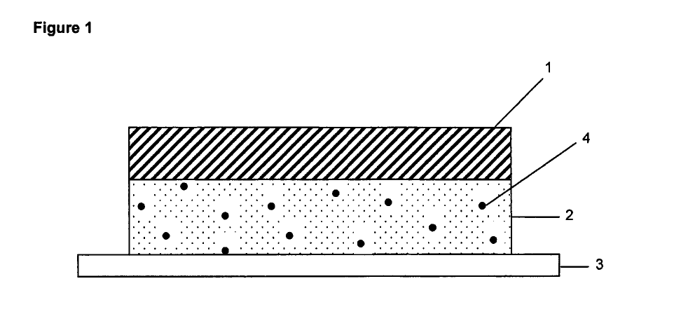

The preferred constitution of the transdermal therapeutic system according to the invention in its simplest development is shown in FIG. 1. At the end of the transdermal therapeutic system that in application is opposite to the skin there is the back layer (1). On the side of the back layer (1) that in use faces the skin there is the pressure-sensitive adhesive matrix layer (2) that is also referred to as reservoir. The preferred transdermal therapeutic system according to the invention is a suspension patch, i.e. the active ingredient is partially undissolved suspended in the pressure-sensitive adhesive matrix layer. In FIG. 1 there are shown the active ingredient particles (4).

It is essential for the preferred transdermal therapeutic systems according to the invention that the pressure-sensitive adhesive matrix layer contains so much active ingredient that a part of the active ingredient is present in an undissolved form, that is as active ingredient particles.

In the preparation of the transdermal therapeutic system according to the invention the active ingredient is preferably employed in the micronized form wherein more than 90% of the particles are smaller than 50 μm, preferably smaller than 25 μm. Also in the matrix layer of the transdermal therapeutic system the active ingredient is present in the micronized form, but rearrangement reactions in the preparation and storage of the transdermal therapeutic system can result in variations of the particle size. Also within the transdermal therapeutic system preferably more than 90% of the active ingredient particles are smaller than 100 μm, more preferably smaller than 50 μm, and in particular smaller than 25 μm. For the preparation of the transdermal therapeutic system according to the invention there is preferably used micronized fentanyl having a mean particle size of 1 μm or more, more preferably 2 μm or more. In the employed fentanyl preferably more than 90% of the particles are smaller than 25 μm. These particle sizes preferably are also found in the finished transdermal therapeutic system. In the matrix layer of the transdermal therapeutic system the particle size and the particle size distribution of the active ingredient particles can be best determined by conventional light microscopy. The evaluation is done with conventional computer programs (image-processing systems) that in general are matched to the used microscopes. The particle size relates to the particle diameter, unless otherwise stated or obvious.

As the starting material for the micronized fentanyl there is used the commercially available fentanyl which is per se suitable for clinical application. Typically, such fentanyl has a distribution of the particle size such that 90% of the particles are smaller than 2,500 μm. Preferably, about 90% of the particles are smaller than about 1,000 μm, and/or more preferably 50% of the particles are smaller than about 100 μm.

According to the invention any known micronization process providing the desired particle size can be used. It is preferred to use fentanyl that was micronized by means of a conventional “jet mill”, e.g., a jet mill of the AS type by Hosokawa Alpine AG.

By the micronizing method employed according to the invention the size of the fentanyl particles is preferably adjusted such that the mean particle size is in the above given ranges. It is also preferred that more than 90% of the particles are smaller than 50 μm, more preferably smaller than 25 μm.

For determining the particle size or particle size distribution of the active ingredient, respectively, various methods are available, for example light diffraction methods (laser diffraction analysis) as used in the devices of Malvern Instruments, e.g. the “Malvern MasterSizer X”, mechanical sieve shaking method, as used by FMC for determining the grain size distribution of their AVICEL PH® products, or air jet sieve analyses that can be carried out for example with an ALPINA® air jet model 200.

Unless otherwise stated the (mean) particle sizes or particle size distributions, respectively, are determined with the laser diffraction analysis method, for example with the Mastersizer 2000 device of Malvern.

If the active ingredient is defined via the indication of the mean particle size and the particle size distribution the micronized active ingredient employed according to the invention preferably has a mean particle size of 20 μm or less and it is preferred that the active ingredient has a grain size distribution (particle size distribution) where less than 10% of the particles have a size of 30 μm or more and less than 10% of the particles have a size of 1 μm or less.

The transdermal therapeutic system according to the invention generally has also a covering layer (3) that covers the pressure-sensitive adhesive matrix layer and has to be removed before use and application of the patch.

In the most preferred embodiment the transdermal therapeutic system according to the invention exclusively consists of the back layer (1) and the pressure-sensitive adhesive matrix layer (2) that contains the active ingredient and the covering layer (3) to be removed before use.

It is also possible that the transdermal therapeutic system on the pressure-sensitive adhesive matrix layer (2) has an additional adhesive layer or an overtape that is for improving the adhesion of the transdermal therapeutic system according to the invention to the skin of the patient. An overtape is an additional adhesive layer that is larger than the active ingredient-containing layer or the actual TTS, respectively, and thus improves the adhesion. However, according to the invention it has been found that such an additional adhesive layer or overtape is not required in the transdermal therapeutic system according to the invention and therefore, according to the invention a transdermal therapeutic system is preferred that has no such additional adhesive layer or overtape, respectively. Such an adhesive layer can consist of various pressure-sensitive adhesives known as such, such as e.g. also polyisobutylene, polysilicone, or polyacrylate.

It is also possible that on the pressure-sensitive adhesive matrix layer (2) a membrane is applied that controls the release of the active ingredient. According to the invention it has again been found that in the constitution of the transdermal therapeutic system according to the invention such a membrane is not required, however it can of course be applied (for example for increasing safety). Suitable membranes are known in the prior art and can be constituted for example on the basis of polypropylene or polyethylene vinylacetate, for example the membrane can be a micro-porous polypropylene foil. Suitable membranes are disclosed in WO 2009/130039 on page 5 the disclosure of which is insofar incorporated herein by reference.

However, since in the transdermal therapeutic systems according to the invention such a membrane is not mandatory the transdermal therapeutic systems according to the invention preferably have no such membrane. If such a membrane is still present there is another adhesive layer on it in order that the transdermal therapeutic system adheres to the skin.

In the particularly preferred embodiment of the transdermal therapeutic systems according to the invention thus the transdermal therapeutic systems exclusively consist of the back layer (1), the pressure-sensitive adhesive matrix layer (2) containing the active ingredient and the stripping layer (3) that must be removed before using the transdermal therapeutic system and additional adhesive layers or a membrane controlling the delivery of the active ingredient are not present.

In a preferred embodiment the back layer (1) of the transdermal therapeutic system is occlusive (that is dense). Such back layers are known in the prior art and may for example consist of polyolefines, in particular polyethylene, polyesters or polyurethanes. Also layers containing several different polymers arranged one upon the other may be employed advantageously as back layers in the transdermal therapeutic systems according to the invention. A suitable material for the back layer is for example a polyolefine that is marketed by Mylan Technologies Inc. under the designation Mediflex® 1000. Other suitable materials comprise cellophane, cellulose acetate, ethyl cellulose, plasticizer-containing vinyl acetate vinyl chloride copolymers, ethylene vinyl acetate copolymers, polyethylene terephthalate, nylon, polyethylene, polypropylene, polyvinylidene chloride, ethylene methacrylate copolymers, paper which optionally can be coated, textile fabrics and polyester films such as polyethylene terephthalate films. Particularly preferred are aluminum films and polymer metal composite materials. The thickness of the back layer is, e.g. in the range of 10 μm to 80 μm, as common in the state of the art, and in the examples a back layer having a nominal thickness of about 10-55 μm was employed.

On the back layer of the patch there can be a covering layer, as is known in the prior art. Preferably, the covering layer lies loose on the back layer and is kept by electrostatic forces. Such covering layers are for example described in EP 1 097 090 to which insofar fully reference is made. The covering layer is non-stick, e.g., siliconized or fluorinated at least on the side lying on the back layer.

The stripping layer (3) lying on the pressure-sensitive adhesive matrix layer is usually also referred to as release liner. Typically, it is applied to the pressure-sensitive adhesive matrix layer to prevent that the pressure-sensitive adhesive matrix layer for example sticks together with the package and is removed before using the transdermal therapeutic system. Said stripping layer is preferably made of a polymeric material that optionally may also be metalized. Examples of preferably employed polymeric materials are poly-urethanes, polyvinyl acetate, polyvinylidene chloride, polypropylene, polycarbonate, poly-styrene, polyethylene, polyethylene terephthalate, polybutylene terephthalate as well as optionally papers which are surface-coated with corresponding polymers. More preferably, the stripping layer is coated with a fluoropolymer or siliconized on one or both sides. Preferred are commercial fluoropolymer-coated or siliconized polyester films such as the trade products siliconized on one side Primeliner 75 μm or 100 μm (Loparex, N L) as well as the trade products fluoropolymer-coated on one side Scotchpak 1022 or Scotchpak 9742 of 3M.

Important according to the invention is the composition of the pressure-sensitive adhesive matrix layer containing the active ingredient. The polyisobutylenes used for the matrix layer have to be very carefully selected to guarantee an active ingredient release which is sufficient and as complete as possible, but at the same time ensure a sufficient adhesion without notable skin irritations on the skin over a period of at least three days and minimization of cold flow also during storage. The choice of the corresponding polymers is complicated by the fact that the transdermal therapeutic systems are stored at another temperature than they are used. The transdermal therapeutic systems are stored at room temperature and then, with the pressure-sensitive adhesive layer applied to the human skin that has a temperature of about 32° C. At this temperature the transdermal therapeutic systems then must sufficiently adhere and exhibit an active ingredient release which is sufficient and as complete as possible.

According to the invention transdermal therapeutic systems have surprisingly been found in which the cold flow is markedly low in particular also at the application temperature (skin temperature=32° C.) and decreases with increasing temperature. However, the transdermal therapeutic systems according to the invention also at the temperatures at which the transdermal therapeutic systems are often stored have a particularly low cold flow.

According to the invention it has now surprisingly been found that the temperature dependency of the storage modulus of a polyisobutylene has a decisive influence on the properties of a transdermal therapeutic system. In particular is has been found that an outstanding active ingredient release up to an extremely low content of residual active ingredient at simultaneously excellent and long-lasting adhesion of the pressure-sensitive adhesive matrix layer to the skin and substantial absence of cold flow during the storage of the transdermal therapeutic system can be achieved if a mixture of two polyisobutylenes is used as the pressure-sensitive adhesive matrix material one of which, herein referred to as polyisobutylene (A), in the temperature range of from 10 to 40° C., preferably from 10 to 60° C. and in particular from 0 to 80° C. virtually exhibits no temperature dependency of the storage modulus G′, whereas the second polyisobutylene, herein referred to as polyisobutylene (B), in this temperature range has a storage modulus G′ that continuously decreases with increasing temperature. Due to this surprising finding, then the transdermal therapeutic system according to the invention has been developed.

The storage modulus G′ as well as the loss modulus G″ are parameters known to the skilled person that characterize a viscoelastic substance. It is assumed that the intensity of the elastic portion of a viscoelastic polymer is described by the storage modulus G′ and the intensity of the viscous portion of a viscoelastic polymer by the loss modulus G″. The quotient from loss modulus G″ and storage modulus G′ G″/G′ is referred to as loss tangent tan δ. The frequency for the measurement of G′ and G″ in the present invention is kept constant at 10 rad/second. In the scope of the present application the storage modulus G′ and the loss modulus G″ were determined with a Rheometrics RDA-III device of TA-Instruments, Newcastle, Del., USA (parallel plates, 8 mm in diameter, distance 2-2.5 mm). The determination of G′ and G″ is performed at low displacement stresses in the measurement in the linear viscoelastic region and thus is independent of the stress. For the rest, G′ and G″ are determined according to the regulations of DIN EN ISO 6721-1:2011.

Polyisobutylenes are known in the prior art and are offered by a number of companies also in the form of solutions among other things. The polyisobutylenes can contain conventional stabilizing agents or preservatives. The term “polyisobutylenes”, as used in the scope of this application, comprises polyisobutylenes containing such conventional preservatives and/or stabilizing agents or not.

The storage modulus G′ in addition to the molecular weight distribution is a typical parameter for the characterization of the polyisobutylenes. Polyisobutylenes that are characterized by certain storage moduli G′ are commercially available. The poly-isobutylene solutions used in the examples of the present application were obtained from Henkel Corporation, Bridgewater, USA, but of course also rival products can be used. The indication of the desired characteristic of the storage modulus is sufficient for the manufacturer of the polyisobutylenes to provide a corresponding polyisobutylene. Of course, the storage modulus is determined on the polyisobutylene after the solvent has been removed.

The transdermal therapeutic system according to the invention has a pressure-sensitive adhesive matrix layer containing a mixture of a polyisobutylene A and a polyisobutylene B.

Polyisobutylene A is characterized by its storage modulus G′ that is substantially constant in the temperature range of from 10 to 40° C., preferably from 10 to 60° C. and in particular from 0 to 80° C., that is in said temperature range the storage modulus G′ has a plateau. By “substantially constant” it is understood according to the invention that in plotting the values of the storage modulus G′ (as logarithm) against the temperature in the temperature range of from at least 10 to 40° C., preferably from 10 to 60° C. and in particular from 0 to 80° C. a flat plateau is shown. In a preferred embodiment of the transdermal therapeutic system according to the invention “substantially constant” means that in the given temperature range none of the values for the storage modulus G′ deviates downward by more than 50% (absolute, i.e. non-logarithmic), more preferably by more than 25% (absolute) and in particular by more than 15% (absolute) from the value of the storage modulus at 40° C. and that none of the values for the storage modulus G′ deviates upward by more than 100% (absolute), preferably by more than 50% (absolute), more preferably by more than 25% (absolute).

The absolute value of the plateau in the range of from 10 to 40° C., preferably from 10 to 60° C. and in particular from 0 to 80° C. for polyisobutylene A is preferably in the range of from 5×10⁴ Pascal to 5×10⁶ Pascal, in particular in the range of from 10⁵ to 10⁶ Pascal and most preferably in the range of from 10⁵ Pascal to 5×10⁵ Pascal. The molecular weight distribution of the polyisobutylene A is not relevant for the present invention, as long as the values for the storage modulus G′ are met.

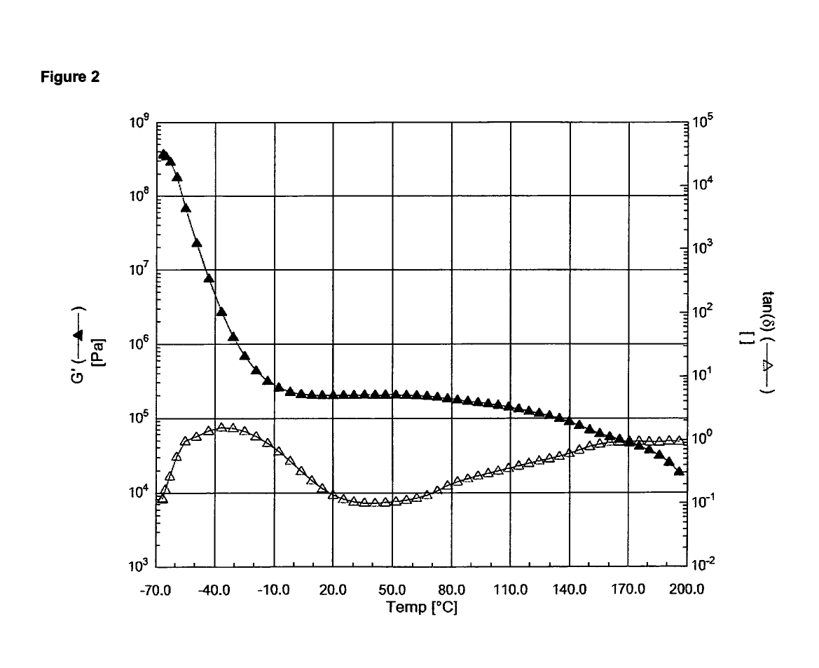

Polyisobutylene A may be prepared for example by mixing two or more polyisobutylenes, so that a polyisobutylene having the desired characteristic for the storage modulus results. However, preferably polyisobutylene A is an individual polyisobutylene. One example for a suitable polyisobutylene A is the product Durotak 87-625A of Henkel Corporation, Brigewater, USA. The characteristic of the storage modulus G′ (together with the characteristic of the quotient from the loss modulus G″ and the storage modulus G′ that is referred to as tan 5 or also as loss tangent) for the commercial product Durotak 87-625A is shown in FIG. 2.

While the product Durotak 87-625A is preferred as the polyisobutylene A according to the present invention, of course also the corresponding products of other manufacturers or of the same manufacturer can be used in which the temperature dependency of the storage modulus is in the range according to the invention.

In the pressure-sensitive adhesive matrix layer of the transdermal therapeutic system according to the invention the polyisobutylene A is present in a mixture with a second polyisobutylene that is herein referred to as polyisobutylene B.

Also polyisobutylene B is characterized by its storage modulus G′. Unlike polyiso-butylene A in the polyisobutylene B the storage modulus must continuously decrease in the range of from 10 to 40° C., preferably from 10 to 60° C. and in particular from 0 to 80° C., and the value for the storage modulus G′ of the polyisobutylene B at the lower temperature of the respective range is markedly higher than at the higher temperature of the respective range. The storage modulus G′ of the polyisobutylene B in general at 0° C. is at least twice as high (absolute) as at 80° C., preferably the storage modulus G′ at 0° C. is at least five times as high (absolute) as at 80° C. and particularly preferred the storage modulus G′ of the polyisobutylene B at 0° C. is at least ten times as high (absolute) as at 80° C. Generally, also in the polyisobutylene B the storage modulus G′ at a temperature of 10° C. is at least twice as high (absolute), preferably at least three times as high (absolute) as at a temperature of 40° C.

The absolute value of the storage modulus G′ at 0° C. in the polyisobutylene B is preferably in the range of from 5×10⁴ Pascal to 5×10⁶ Pascal, in particular in the range of from 5×10⁴ Pascal to 10⁶ Pascal and especially preferred in the range of from 10⁵ to 10⁶ Pascal. The absolute value of the storage modulus G′ at 80° C. in the polyisobutylene is preferably in the range of from 5×10² Pascal to 5×10⁴ Pascal, more preferably in the range of from 5×10² Pascal to 10⁴ Pascal and in particular in the range of from 10³ to 10⁴ Pascal.

The absolute value of the storage modulus G′ in the polyisobutylene B at 10° C. is preferably in the range of from 2×10⁴ Pascal to 10⁶ Pascal, in particular in the range of from 5×10⁴ Pascal to 10⁶ Pascal. The absolute value of the storage modulus G′ at 40° C. in the polyisobutylene B is preferably in the range of from 5×10³ Pascal to 2×10⁵ Pascal, more preferably in the range of from 10⁴ Pascal to 10⁵ Pascal. The absolute value of the storage modulus G′ at 60° C. in the polyisobutylene B is preferably in the range of from 5×10² Pascal to 10⁵ Pascal, more preferably in the range of from 5×10³ Pascal to 5×10⁴ Pascal. As with the polyisobutylene A, also for the polyisobutylene B the molecular weight distribution is not relevant for the present invention, as long as the storage modulus G′ has the temperature dependency that is required according to the invention.

Furthermore it has been shown that for solving the problem according to the invention such transdermal therapeutic systems are preferred that have a certain ratio of storage modulus to loss modulus. In this preferred embodiment the transdermal therapeutic systems according to the invention can additionally be given by the value of the loss tangent 8 at 30° C. So, in the polyisobutylene A the loss tangent 8 at 30° C. is preferably in the range of from 10⁻² to 5×10⁻¹, more preferably in the range of from 5×10⁻² to 5×10⁻¹. In the polyisobutylenes B according to the invention the loss tangent 8 at 30° C. is preferably in the range of from more than 5×10⁻¹ to 10, in particular in the range of from 6×10⁻¹ to 5. That is, in transdermal therapeutic systems according to the invention the loss tangent in the polyisobutylene B at 30° C. is preferably higher than in the polyisobutylene A.

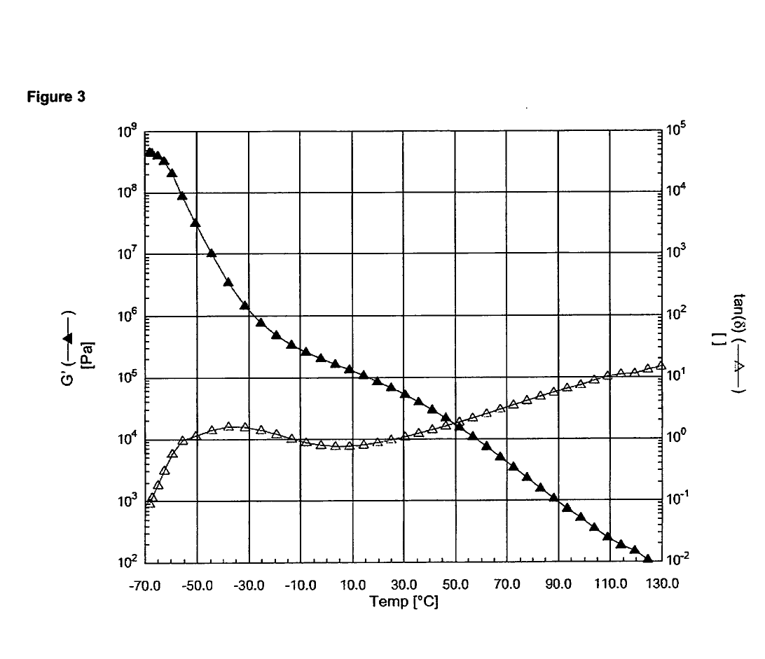

As with the polyisobutylene A, also the polyisobutylene B can be prepared for example by mixing two or more polyisobutylenes, what results in a polyisobutylene having the desired characteristic for the storage modulus G′. Preferably, the polyisobutylene B is an individual polyisobutylene. One example of a suitable polyisobutylene B is the Durotak 87-626A product from Henkel Corporation, Bridgewater, USA. The characteristic of the storage modulus G′ (together with the characteristic of the quotient from the loss modulus G″ and the storage modulus G′, referred to as tan 5 (or loss tangent)) for the commercial product Durotak 87-626A is shown in FIG. 3.

While the Durotak 87-626A product according to the present invention is preferred as the polyisobutylene B, of course also corresponding products of other manufacturers or the same manufacturer can be used wherein the temperature dependency of the storage modulus is in the range according to the invention.

Since the pressure-sensitive adhesive matrix layer of the transdermal therapeutic system according to the invention contains a mixture of the polyisobutylene A and the polyisobutylene B and the polyisobutylene A and/or the polyisobutylene B preferably consist of one polyisobutylene each or a mixture of at least two polyisobutylenes of different average molecular weights each, the pressure-sensitive adhesive matrix of the transdermal therapeutic system according to the invention can have two, three, four or more polyisobutylenes of different average molecular weights.

If in the context of the present description an average molecular weight is mentioned by that a mean weight average molecular weight is meant unless otherwise explicitly stated or resulting from the circumstances.

According to the invention it is assumed that the individual polyisobutylene has a molecular weight distribution with a single peak without shoulder corresponding to the molecular weight distribution obtained in the polymerization of the polyisobutylene from the individual monomers. When mixing two polyisobutylenes of different molecular weights prepared by simple polymerization according to the invention this is a mixture of two poly-isobutylenes. According to the invention a mixture of two polyisobutylenes is characterized in that the molecular weight distribution of the mixture of two polyisobutylenes has two peaks at two different molecular weights or the molecular weight distribution shows one peak with one shoulder (if the average molecular weights of the mixed polyisobutylenes are too close to each other, so that no two separate peaks are shown).

In the transdermal therapeutic system according to the invention the pressure-sensitive adhesive matrix layer consists of a mixture of a polyisobutylene A and a polyisobutylene B that differ by the temperature dependency of their storage moduli. In general, polyiso-butylene A also has another average molecular weight than the polyisobutylene B. Thus, in general the molecular weight distribution of the mixture of polyisobutylene A and poly-isobutylene B will have at least two peaks one of which goes back to polyisobutylene A and the other goes back to polyisobutylene B. It is also possible that polyisobutylene A and/or polyisobutylene B on the other hand were prepared of at least two polyisobutylenes of different molecular weights. However, in the transdermal therapeutic system according to the invention the pressure-sensitive adhesive matrix layer is preferably constituted of two polyisobutylenes with each polyisobutylene in the pressure-sensitive adhesive matrix layer of the transdermal therapeutic system according to the invention showing a separate peak or a shoulder in the molecular weight distribution.

If the polyisobutylenes A or B each are mixtures of polyisobutylenes the individual poly-isobutylenes from which the polyisobutylene A and polyisobutylene B consist of are selected such that the desired temperature dependency of the storage modulus for poly-isobutylene A or B, respectively, is obtained.

The molecular weight distribution of a polyisobutylene or a mixture of several poly-isobutylenes in the prior art can be determined by gel permeation chromatography (GPC), for example against a polystyrene standard.

The amount of polyisobutylene B in the pressure-sensitive adhesive matrix layer in relation to the amount of polyisobutylene A in the pressure-sensitive adhesive matrix layer can be suitably adjusted by the skilled person. Generally, the weight ratio of polyisobutylene A:polyisobutylene B in the matrix layer is in the range of from 10% (A):90% (B) to 60% (A):40% (B), preferably 20% (A):80% (B) to 50% (A):50% (B), more preferably 20% (A):80% (B) to 40% (A):60% (B), and in particular 25% (A):75% (B) to 35% (A):65% (B), each based on the total weight of the two polyisobutylenes, and however, the content of polyisobutylene B in the matrix layer is preferably higher than the content of polyisobutylene A. The amount of polyisobutylene A in the matrix layer is preferably in the range of from 10 to 25%, more preferably in the range of from 10 to 20%, in particular in the range of from 12 to 20%, in particular from 16 to 18%, each as weight percentage based on the total weight of the matrix layer. The content of polyisobutylene B in the matrix layer is preferably in the range of from 30 to 60%, more preferably in the range of from 30 to 50% and in particular in the range of from 40 to 45%, each as weight peicentage based on the total weight of the matrix layer.

The matrix layer of the transdermal therapeutic system according to the invention contains the active ingredient as well as the polyisobutylene A and the polyisobutylene B. Additionally, the matrix layer preferably also contains a permeation enhancer. As the permeation enhancer any permeation enhancer known in the prior art may be used for the active ingredient fentanyl. Particularly preferred according to the invention a carboxylic acid ester and here in particular a fatty acid ester is used. Particularly preferred is the myristic acid isopropyl ester (isopropyl myristate) and the oleic acid oleyl ester (oleyl oleate). It has surprisingly been shown that by using the matrix according to the invention the permeation enhancer isopropyl myristate is particularly suitable to provide a transdermal therapeutic system having the desired properties.

The content of permeation enhancer in the matrix layer of the transdermal therapeutic system according to the invention is not particularly limited, but generally it is in the range of from 2 to 20% by weight, based on the total weight of the matrix layer, particularly preferred in the range of from 5 to 15% by weight, for example at 10% by weight, based on the total weight of the matrix layer. When using isopropyl myristate as the permeation enhancer the range of 8 to 15% by weight, based on the total weight of the matrix layer according to the invention, turned out as particularly advantageous.

More preferably, the pressure-sensitive adhesive matrix layer of the transdermal therapeutic system according to the invention also contains a tackifier. As the tackifier a polybutene is particularly suitable, however, another tackifier such as rosin, terpene resin or petroleum resin suitable for polyisobutylene may be used. However, polybutene is preferred as the tackifier, in particular polybutene having a number-average molecular weight in the range of from 700 to 6000, in particular of 900 to 4000, such as the Indopol H-1900 product having a mean number-average molecular weight of 2500. The poly-butene is an isobutylene/butene copolymer.

Moreover, as the tackifier a hydrogenated or non-hydrogenated rosin, in particular a hydrogenated rosin is preferred. As an example for this the commercial product Foral® 105-E of Eastman Chemical Middelburg BV, Den Haag, Netherlands can be mentioned.

The content of tackifier, in particular of polybutene in the matrix layer of the transdermal therapeutic system according to the invention in general is 0 to 40% by weight, preferably 5 to 35% by weight, in particular 10 to 30% by weight, e.g. about 25% by weight, each based on the total weight of the matrix layer.

When using isopropyl myristate as the permeation enhancer it has surprisingly found to be advantageous to use a polybutene having a number-average molecular weight in the range of from 1800 to 2800, namely particularly advantageous in the range of from 22 to 28% by weight, based on the total weight of the matrix layer according to the invention.

Moreover, it has surprisingly been shown that with the ratios of Indopol 1900, poly-isobutylene A and polyisobutylene B that are preferred according to the invention in the transdermal therapeutic system the cold flow at typical storage temperatures of less than 20° C. and after application to the skin (i.e. at temperatures >30° C.) is particularly low.

According to the invention there are preferably no further components in the matrix layer of the transdermal therapeutic system according to the invention. Thus, according to the invention the matrix layer of the transdermal therapeutic system according to the invention preferably consists of active ingredient, polyisobutylene A, polyisobutylene B, permeation enhancer (in particular a carboxylic acid ester, preferably isopropyl myristate or oleyl oleate, particularly preferred isopropyl myristate), and tackifier (particularly preferred poly-butene, in particular Indopol H-1900).

The matrix layer of the transdermal therapeutic system according to the invention particularly preferred is free from gel-forming agents.

The matrix of the transdermal therapeutic system according to the invention preferably has no branched higher chain alcohol, particular preferred no higher chain alcohol at all, wherein by a branched higher chain alcohol or a higher chain alcohol an alcohol having 5 or more carbon atoms is meant.

In a preferred embodiment the polyisobutylene A for example has a mean viscosity-average molecular weight of ca. 1,100,000 g/mol and the polyisobutylene B has a mean viscosity-average molecular weight of ca. 30,000 to ca. 60,000 g/mol, preferably ca. 40,000 or 55,000 g/mol. In this embodiment, the patch further contains a tackifier and a permeation enhancer, in particular the Indopol H 1900 tackifier, that is a polybutene having a number-average molecular weight of about 2500 g/mol, and as the permeation enhancer in particular either oleyl oleate or isopropyl myristate, wherein the polybutene preferably is present in an amount of from 23 to 28% by weight and the isopropyl myristate or oleyl oleate, respectively, is present in an amount of from 8 to 15% by weight.

In the transdermal therapeutic system according to the invention the matrix layer preferably has an amount of fentanyl or an analogue thereof sufficient to induce analgesia in a human being and maintain it for at least three days, preferably three days (based on the point of administration of the patch). It is also preferred for the matrix layer to contain an amount of fentanyl or an analogue thereof sufficient to induce analgesia and maintain it for a period of at least three days, in particular three to seven days.

The absolute amount of active ingredient to be employed depends on various factors, in particular the size of the patch to be employed and the duration of application. Preferably, the transdermal therapeutic system contains the active ingredient (in particular fentanyl) in an amount of from 3 to 15% by weight, particularly preferred of from 3 to 10% by weight, in particular of from 4 to 8% by weight, 4 to 6% by weight or 5 to 6% by weight, for example about 6% by weight, each based on the total weight of the matrix layer. Depending on the exact composition of the matrix layer and the amount of active ingredient used the active ingredient may either be present completely dissolved in the matrix layer or there are individual active ingredient particles undissolved in the matrix layer. If individual active ingredient particles are undissolved in the matrix layer one speaks of a suspension patch. A suspension patch is preferred according to the invention and shown in FIG. 1, wherein reference number 4 indicates the solid active ingredient particles in the matrix layer.

From the preceding explanations preferred embodiments of the transdermal therapeutic system according to the invention result wherein the matrix layer has a composition as summarized in the table below.

| | % by | % by | % by | % by | % by |
|---|---|---|---|---|---|
| COMPONENT | weight | weight | weight | weight | weight |
| Fentanyl | 3-10 | 4-8 | 4-6 | 5.0-6.0 | 6 |
| Permeation Enhancer | 5-15 | 8-15 | 8-12 | 9-11 | 10 |
| Polyisobutylene B | 20-60 | 30-50 | 35-50 | 40-45 | 41.2 |
| Polyisobutylene A | 5-30 | 8-25 | 12-20 | 16-18 | 17.6 |
| Tackifier | 15-45 | 15-40 | 20-35 | 23-28 | 25.2 |

| | % by | % by | % by | % by | % by |
|---|---|---|---|---|---|
| COMPONENT | weight | weight | weight | weight | weight |
| Fentanyl | 3-10 | 4-8 | 4-6 | 5.0-6.0 | 6 |
| Isopropyl Myristate | 5-15 | 8-15 | 8-12 | 9-11 | 10 |
| Polyisobutylene B | 20-60 | 30-50 | 35-50 | 40-45 | 41.2 |
| (particularly preferred | | | | | |
| Durotak 87-626A) | | | | | |
| Polyisobutylene A | 5-30 | 8-25 | 12-20 | 16-18 | 17.6 |
| (particularly preferred | | | | | |
| Durotak 87-625A) | | | | | |
| Polybutene having a | 15-45 | 15-40 | 20-35 | 23-28 | 25.2 |
| number-average | | | | | |
| molecular weight | | | | | |
| of about 2500 | | | | | |

The weight percentages in the preceding tables each relate to the total weight of the matrix layer. The weight per unit area of the matrix layer according to the invention is preferably in the range of from 20 g/m² to 100 g/m², in particular in the range of from 30 g/m² to 70 g/m², each based on the weight per unit area of the dried matrix layer.

In a preferred embodiment the present invention relates to a transdermal system, in particular for the alleviation of pain during the intended wearing time, as described above, in which the matrix layer, after application to the skin for a duration of the intended wearing time of typically 1 to 7 days, preferably 3 to 7 days, in particular 3 days, has a residual content of active ingredient of typically below 35%, preferably below 25%, more preferably below 20%, still more preferably below 15%, in particular below 10%, e.g. below 8%, 7%, 6%, or 5% of the initial content of active ingredient. Typically, the residual content of active ingredient is in the range of from below 35% to 10%, preferably below 25% to 2%, more preferably in the range of from below 20% to 3%, more preferably in the range of from below 15% to 4%, in particular below 10% to 5% of the initial content of active ingredient. The initial content of active ingredient relates to the absolute amount of active ingredient to be employed in the matrix layer as defined above. The residual content of active ingredient (in %) results from the quotient from the absolute amount of residual content of active ingredient of the matrix layer and the absolute amount of active ingredient to be employed. The absolute amount of the residual content of active ingredient of the matrix layer is the amount of active ingredient that remains in the transdermal system after application to the skin for the intended wearing time, typically 1 to 7 days, preferably 3 to 7 days, e.g. 3 days. The absolute amount of the residual content of active ingredient of a transdermal system can be determined with methods known in the prior art. So, for example the matrix layer of the used transdermal system can be dissolved in a suitable solvent and the total amount of active ingredient in the solution can be determined, for example in a chromatographic way.

It has surprisingly been shown that in particular by choosing the above-mentioned polymers in the matrix layer despite a low active ingredient input an excellent active ingredient flow and thus high delivery rates over the intended period of application of the transdermal system up to very low residual contents of active ingredient can be realized. Preferably, the transdermal therapeutic system according to the present invention has a delivery rate of the active ingredient that corresponds to that of a transdermal therapeutic system that has been approved by at least one medicine agency. Such typical delivery rates are in the range of from about 12.5 μg/h to about 100 μg/h, preferably 12.5 μg/h, 25 μg/h, 50 μg/h, 75 μg/h and/or 100 μg/h. In one embodiment the transdermal therapeutic system has a delivery rate of at least 100 μg/h or more, preferably at least 100 μg/h or more to about 300 μg/h, in particular about 150 μg/h to about 250 μg/h, e.g. about 200 μg/h or 250 μg/h. Preferably, the delivery rates are achieved at an active ingredient load that in comparison to the known, in particular approved transdermal therapeutic systems is low (absolute amount of active ingredient in the transdermal therapeutic system). By the low residual content of active ingredient achieved according to the invention at high delivery rats thus not only unnecessary costs by unused active ingredient remaining in the transdermal system are avoided, but also the hazard and abuse potential by worn transdermal systems that still have an residual content of active ingredient is reduced. Thus, the present invention relates to worn transdermal therapeutic systems as defined above that have the above-mentioned content of residual active ingredient after use.

The transdermal therapeutic system according to the invention is prepared in accordance with methods basically known in the prior art. For that, the active ingredient is dispersed in the permeation enhancer. Polyisobutylene A and polyisobutylene B are also dissolved in a suitable solvent, in particular a liquid alkane, in particular an alkane having 5 to 7 carbon atoms, such as e.g. heptane. The dissolved polyisobutylene is mixed with the dispersed fentanyl under stirring. Subsequently, the tackifier is optionally added and stirring is continued until a homogeneous coating mass forms. The homogeneous coating mass is then applied to the stripping layer and dried under heating, so that the residual content of solvent is as low as possible. Preferably, the residual content of solvent is less than 1%, in particular less than 0.5%. The dried matrix is coated with the back layer what results in a laminate. From this laminate patches of the suitable size are punched out.

The transdermal therapeutic systems described herein may be prepared according to the preceding method according to the invention and thus the invention relates to a method for the preparation of the transdermal therapeutic systems according to the invention and transdermal therapeutic systems obtainable according to said method.

Moreover, the present invention also relates to the use of a mixture of polyisobutylene A and polyisobutylene B as described above for the preparation of a matrix layer of a transdermal therapeutic system or the use of such a matrix layer for administering fentanyl or an analogue of the fentanyl as described above, wherein the matrix layer of the transdermal therapeutic system after application to the skin for the intended wearing time of typically 1 to 7 days, preferably 3 to 7 days, in particular 3 days, has a residual content of active ingredient as defined above for the matrix, in particular below 35%, preferably below 25%, more preferably below 20%, still more preferably below 15%, in particular below 10%, for example below 8%, 7%, 6%, or 5% of the initial content of active ingredient. Also, the present invention relates to the use of such a mixture of polyiso-butylene A and polyisobutylene B for the preparation of a matrix layer of a transdermal therapeutic system or the use of such a matrix layer in a transdermal therapeutic system, that in particular after use is protected from abuse or misuse. Also, the present invention relates to the use of a mixture of polyisobutylene A and polyisobutylene B for reducing the size of a transdermal therapeutic system at a substantially constant release, in particular the above-mentioned delivery rates. In particular, according to the invention a mixture of polyisobutylene A and polyisobutylene B for the preparation of a matrix layer of a transdermal therapeutic system is used or such a matrix layer is employed in a transdermal therapeutic system to reduce the size of the transdermal therapeutic system at a release profile that in comparison to a commercially available transdermal therapeutic system such as for example Matrifen® and Durogesic DTrans® is constant, that is bioequivalent. Therefore, according to the invention a transdermal therapeutic system is provided that with low size is bioequivalent to the market product, in particular the commercially available Matrifen® or Durogesic DTrans®. Employing small transdermal therapeutic systems is desired in particular for cosmetic reasons.

Finally, the present invention relates to the use of a mixture of polyisobutylene A and polyisobutylene B as described above for the preparation of a matrix layer of a transdermal therapeutic system or the use of such a matrix layer in a transdermal therapeutic system having a delivery rate as defined above, in particular of at least 200 μg/h, e.g. at least 250 μg/h. Preferably, the transdermal therapeutic system according to the invention has a size of less than 80 cm², more preferably less than 60 cm², still more preferably less than 50 cm², in particular less than 45 cm².

According to the invention it has further been found that the following transdermal system having a pressure-sensitive adhesive matrix layer on the basis of a specific polyacrylate also has the outstanding properties according to the invention, in particular the fast and substantially complete release.

Thus, the present invention in this embodiment relates to a transdermal therapeutic system for the administration of an active ingredient through the skin comprising or consisting of
a) a back layer,
b) a pressure-sensitive adhesive matrix layer containing the active ingredient; and
c) a stripping layer (release liner),
   wherein the active ingredient is fentanyl or an analogue of the fentanyl selected from alfentanil, lorfentanil, lofentanil, remifentanil, and trefentanil or a salt of one of these active ingredients, preferably fentanyl and is preferably contained in the matrix layer in an amount of 8% by weight to 12% by weight, based on the total weight of the matrix layer, more preferably about 10% by weight, wherein the matrix layer as the pressure-sensitive adhesive polymer contains an acrylate-vinylacetate copolymer in an amount of ca. 82-88% by weight, preferably about 85% by weight, based on the total weight of the matrix layer, as well as about 5% by weight of a polymer regulator, preferably PVP, wherein the pressure-sensitive adhesive matrix layer contains no undissolved active ingredient and wherein the acrylate-vinylacetate copolymer is constituted of about 68% of 2-ethylhexyl-acrylate, about 27% of vinylacetate, and about 5% of 2-hydroxyethylacrylate as well as optionally small amounts (<0.5%) of glycidylmethacrylate as cross-linking agent (all percentages as weight percentages). Preferred are the commercial products DURO-TAK 87-2287, DURO-TAK 87-4287 or DURO-TAK 87-3916. Preferably, the commercial product Polyvidon 25 is used as the PVP.

The transdermal therapeutic system according to the embodiment based on a matrix layer on the basis of the specific polyacrylate with respect to the constitution of back layer (1), matrix layer (2), optionally the covering layer (3) to be removed before use and stripping layer corresponds to the above-described embodiment of the transdermal therapeutic system with a matrix layer on the basis of a polyisobutylene. The above-described preferred embodiments for the back layer (1), stripping layer (3) correspondingly apply to the transdermal system containing a matrix layer on the basis of a polyacrylate. Also, the above-described permeation enhancer, in particular isopropyl myristate or oleyl oleate may be used in the above-mentioned amounts.

The following example explains the invention.

A transdermal therapeutic system with a pressure-sensitive adhesive matrix layer of the following composition:
6% of Fentanyl
10% of Isopropyl Myristate
41.2% of Durotak 87-626A
17.6% of Durotak 87-625A and
25.2% of Indopol 1900
   was prepared as follows.

0.6 g of fentanyl were dispersed in 1.0 g of isopropyl myristate. The batch was stirred to a homogeneous suspension. 5.58 g of Durotak 87-626A (solids content 73.71%) and 14.48 g of Durotak 87-625A (solids content 12.18%) were added to the batch of fentanyl and iso-propyl myristate under stirring over a period of 20 minutes. Subsequently, 2.52 g of poly-butene were added to the residual mixture.

It was stirred until a homogeneous coating mass is formed. Said homogeneous coating mass was applied to a film of the brand Scotchpak 9742 with a thickness of 117 μm. Subsequently, the mixture was gradually heated to 50° C., 60° C., 70° C., and 80° C. and dried. The weight per unit area of the dried matrix layer was ca. 50 g/m². The dried matrix was coated with a back layer of the brand Scotchpak 9723. From the laminate patch fragments of suitable size were punched out.

The thus prepared patches were stored for 8 weeks in a refrigerator at 2° C. to 8° C., at 25° C. and 60% relative humidity and at 40° C. and 75% relative humidity. No notable cold flow occurred in the patches. In all examined patches under all examined conditions the maximum cold flow was below 0.3 mm. As a comparison a transdermal therapeutic system according to example 2 of WO 2011/029948 was prepared and stored for 8 weeks at 25° C./60% humidity and at 40° C./75% humidity. The comparison patches exhibited a distinct cold flow at the stripping assistance and also at the back layer that was substantially higher than the cold flow that occurred in the patch according to the invention.

With the patches according to the invention in vitro studies have been performed. Permeation rates in Franz cells substantially corresponding to the patch of example 2 of WO 2011/029948 occurred.

The patches adhere without notable skin irritations and are locally good tolerated for up to three days on the skin.

Therefore, the transdermal therapeutic systems according to the invention show the same good cutaneous tolerance, the same adhesive capacity, and the same permeation rates as common commercial systems. However, unlike the transdermal therapeutic systems of WO 2011/029948 the transdermal therapeutic systems according to the invention in storage show virtually no cold flow and are also much easier and cost-effectively to be prepared with an even less content of active ingredient and less residual content of active ingredient.

1. A transdermal therapeutic system for the administration of an active ingredient through the skin comprising or consisting of
a) a back layer,
b) a pressure-sensitive adhesive matrix layer containing the active ingredient; and
c) a stripping layer (release liner),
wherein the active ingredient is fentanyl or an analogue of the fentanyl selected from alfentanil, carfentanil, lofentanil, remifentanil, and trefentanil or a salt of one of these active ingredients and
wherein the matrix layer as the pressure-sensitive adhesive polymer contains a mixture of a polyisobutylene A and a polyisobutylene B,
wherein the polyisobutylene A has a storage modulus G′ the value of which is substantially constant in the temperature range of from 10° C. to 40° C. and
wherein the polyisobutylene B has a storage modulus G′ the value of which continuously decreases with increasing temperature in the temperature range of from 10° C. to 40° C.,
wherein the storage modulus G′ is measured in the linear viscoelastic range at a frequency of 10 rad/sec using a rheometer with parallel plate geometry and parallel plates; and
wherein the pressure-sensitive adhesive matrix layer contains undissolved active ingredient in the form of active ingredient particles.

2. The transdermal therapeutic system according to claim 1, wherein the active ingredient is fentanyl.

3. The transdermal therapeutic system according to claim 1, wherein for the polyisobutylene A in the temperature range of from 10° C. to 40° C. all values of the storage modulus G′ deviate from the value for the storage modulus G′ at 40° C. by no more than 50%.

4. The transdermal therapeutic system according to claim 1, wherein for the polyisobutylene B the storage modulus G′ at 10° C. is at least two times the storage modulus G′ at 80° C.

5. The transdermal therapeutic system according claim 1, wherein the content of the polyisobutylene A to the polyisobutylene B in the matrix layer is in the range of from 20% (A) : 80% (B) to 40% (A) : 60% (B), each based on the total weight of the polyisobutylenes A and B.

6. The transdermal therapeutic system according to claim 1, wherein the polyisobutylene A and the polyisobutylene B each are individual polyisobutylenes of different average molecular weights.

7. The transdermal therapeutic system according to claim 1, wherein the matrix layer contains a permeation enhancer that optionally is isopropyl myristate or oleyl oleate.

8. The transdermal therapeutic system according to claim 1, wherein the matrix layer contains a tackifier that optionally is a polybutene or hydrogenated or non-hydrogenated rosin ester.

9. The transdermal therapeutic system according to claim 1, characterized in that the amount of active ingredient is sufficient for an application time of 3 days and the active ingredient is present in the matrix layer at a concentration in the range of from 3-15% by weight, based on the weight of the matrix layer.

10. The transdermal therapeutic system according to claim 1, characterized in that in the pressure-sensitive adhesive matrix layer in addition to the active ingredient, the polyisobutylene A, and the polyisobutylene B, only a tackifier, optionally a polybutene or a hydrogenated rosin ester, and a permeation enhancer, optionally isopropyl myristate or oleyl oleate, are present.

11. The transdermal therapeutic system according to claim 10, wherein the tackifier is present in an amount in the range of from 23 to 28% and the permeation enhancer is present in an amount in the range of from 8 to 15%, each based on the total weight of the matrix layer.

12. A method for the preparation of a transdermal therapeutic system according to claim 1, wherein the active ingredient is dispersed in the permeation enhancer,
the polyisobutylene A and the polyisobutylene B each are distributed in a suitable solvent, subsequently both polymer-containing solutions are homogeneously mixed,
the polymer-containing solutions are mixed with the dispersed active ingredient and optionally further components until a uniform mass is formed,
the thus obtained mass is applied to the stripping layer or the back layer,
the solvent is removed,
the back layer or the stripping layer, respectively, is laminated thereon; and
transdermal therapeutic systems of the desired size are cut out or punched out.

13. The transdermal therapeutic system obtained according to the method according to claim 12.

14. The transdermal therapeutic system according to 13 to alleviate pain during an intended wearing time of optionally 3 to 7 days, wherein the matrix layer after application to the skin for the duration of the intended wearing time has a residual content of active ingredient below 35% of the initial content of active ingredient.

15. The transdermal therapeutic system according to claim 14, wherein the transdermal therapeutic system has a delivery rate of the active ingredient that corresponds to that of a transdermal therapeutic system approved by at least one medicine agency.

16. A used transdermal therapeutic system obtained by removing the transdermal therapeutic system according to claim 1 that was applied to the skin for the duration of an intended wearing time of optionally 3 to 7 days.

17. A method of utilizing of the pressure-sensitive adhesive matrix layer in the transdermal therapeutic system as defined in claim 1 for the preparation of the transdermal therapeutic system alleviating pain during an intended wearing time of optionally 3 to 7 days, wherein the matrix layer of the transdermal therapeutic system to be prepared after application to the skin for the intended wearing time has a residual content of active ingredient below 35 of the initial content of active ingredient.

18. A method of utilizing of the pressure-sensitive adhesive matrix layer in the transdermal therapeutic system as defined in claim 1 for the preparation of the transdermal therapeutic system protected from abuse or misuse.

19. A method of utilizing of the pressure-sensitive adhesive matrix layer in the transdermal therapeutic system as defined in claim 1 to reduce the size of the transdermal therapeutic system at a substantially constant release profile.

20. The method according to claim 19, wherein the transdermal therapeutic system is or was a commercially available system, optionally Matrifen® and Durogesic DTrans®.

21. A method of utilizing of the pressure-sensitive adhesive matrix layer in the transdermal therapeutic system as defined in claim 1 for providing the transdermal therapeutic system having a delivery rate of more than 100 μg/h.

22. The method according to claim 21, wherein the transdermal therapeutic system has a size of less than 50 cm².
